(19) United States
(12) Patent Application Publication
(10) Pub. No.: US 2024/0187788 A1
(43) Pub. Date: Jun. 6, 2024

(54) SOUND SIGNAL PROCESSING METHOD, RECORDING MEDIUM, AND SOUND SIGNAL PROCESSING DEVICE

(71) Applicant: Panasonic Intellectula Property Management Co., Ltd., Osaka (JP)

(72) Inventors: Atsushi SAKAGUCHI, Kyoto (JP); Tadayoshi OKUDA, Osaka (JP); Ai AMEMIYA, Tokyo (JP)

(21) Appl. No.: 18/437,540

(22) Filed: Feb. 9, 2024

Related U.S. Application Data
(63) Continuation of application No. PCT/JP22/19710, filed on May 9, 2022.

(30) Foreign Application Priority Data
Aug. 20, 2021 (JP) 2021-135001

(51) Int. Cl. H04R 3/04

(52) U.S. Cl. CPC H04R 3/04

(57) ABSTRACT

A sound signal processing method according to an aspect of the present disclosure includes: adjusting a signal level of a second sound signal (for example, an additional signal) according to a signal level of a specific frequency band in a first sound signal (for example, a sound source signal) corresponding to a first content, the second sound signal corresponding to a second content and including only a component of the specific frequency band; and superimposing and outputting the first sound signal and the second sound signal adjusted.

### CROSS REFERENCE TO RELATED APPLICATIONS

This is a continuation application of PCT International Application No. PCT/JP2022/019710 filed on May 9, 2022, designating the United States of America, which is based on and claims priority of Japanese Patent Application No. 2021-135001 filed on Aug. 20, 2021. The entire disclosures of the above-identified applications, including the specifications, drawings and claims are incorporated herein by reference in their entirety.

### FIELD

The present disclosure relates to a sound signal processing method, a recording medium, and a sound signal processing device.

### BACKGROUND

Patent Literature (PTL) 1 discloses a device for treating dementia or Alzheimer's disease through a combination of auditory stimulation and visual stimulation.

### CITATION LIST

### Patent Literature

PTL 1: Japanese Unexamined Patent Application Publication (Translation of PCT Application) No. 2020-536653

### SUMMARY

### Technical Problem

The present disclosure provides a sound signal processing method and the like that can suitably output sound in a specific frequency band to a target.

### Solution to Problem

A sound signal processing method according to an aspect of the present disclosure includes: adjusting a signal level of a second sound signal corresponding to a second content according to a signal level of a specific frequency band in a first sound signal corresponding to a first content, the second sound signal including a component of the specific frequency band; and superimposing and outputting the first sound signal and the second sound signal adjusted.

In addition, a sound signal processing method according to another aspect of the present disclosure includes: adjusting, to increase, a signal level of a specific frequency band of each of a plurality of first sound signals in a first content including a plurality of sound contents that are different from each other, the plurality of first sound signals corresponding to the plurality of sound contents; correcting each of the plurality of first sound signals adjusted to reduce a phase difference of the specific frequency band among the plurality of first sound signals adjusted, the plurality of first sound signals adjusted corresponding to the plurality of sound contents; and outputting the plurality of first sound signals corrected.

In addition, a recording medium according to one aspect of the present disclosure is a non-transitory computer-readable recording medium having recorded thereon a program for causing a computer to execute the sound signal processing method described above.

A sound signal processing device according to an aspect of the present disclosure includes: a processor and memory, wherein using the memory, the processor: adjusts a signal level of a second sound signal corresponding to a second content according to a signal level of a specific frequency band in a first sound signal corresponding to a first content, the second sound signal including a component of the specific frequency band; and superimposes and outputs the first sound signal and the second sound signal adjusted.

In addition, a sound signal processing device according to another aspect of the present disclosure includes: a processor and memory, wherein using the memory, the processor: adjusts, to increase, a signal level of a specific frequency band of each of a plurality of first sound signals in a first content including a plurality of sound contents that are different from each other, the plurality of first sound signals corresponding to the plurality of sound contents; corrects each of the plurality of first sound signals adjusted to reduce a phase difference of the specific frequency band among the plurality of first sound signals adjusted, the plurality of first sound signals adjusted corresponding to the plurality of sound contents; and outputs the plurality of first sound signals corrected.

### Advantageous Effects

According to the sound signal processing method and the like according to one aspect of the present disclosure, sound in a specific frequency band can be suitably output to a target.

### BRIEF DESCRIPTION OF DRAWINGS

These and other advantages and features will become apparent from the following description thereof taken in conjunction with the accompanying Drawings, by way of non-limiting examples of embodiments disclosed herein.

FIG. 1 is a block diagram showing the configuration of a sound signal processing device according to Embodiment 1.

FIG. 2 is a diagram showing a first sound signal and a sound signal in which a second sound signal is superimposed on the first sound signal.

FIG. 3 is a flowchart showing the processing procedure of the sound signal processing device according to Embodiment 1.

FIG. 4 is a diagram for explaining the calculation result of the envelope calculated from the first sound signal.

FIG. 5 is a flowchart showing the processing procedure of a sound signal processing device according to Variation 1 of Embodiment 1.

FIG. 6 is a flowchart showing the processing procedure of a sound signal processing device according to Variation 2 of Embodiment 1.

FIG. 7 is a diagram for explaining a signal obtained from the calculation result of performing FFT on the first sound signal.

FIG. 8 is a flowchart showing the processing procedure of a sound signal processing device according to Variation 3 of Embodiment 1.

FIG. 9 is a block diagram showing the configuration of a sound signal processing device according to Variation 4 of Embodiment 1.

FIG. 10 is a flowchart showing the processing procedure of the sound signal processing device according to Variation 4 of Embodiment 1.

FIG. 11 is a block diagram showing the configuration of a sound signal processing device according to Variation 5 of Embodiment 1.

FIG. 12 is a flowchart showing the processing procedure of the sound signal processing device according to Variation 5 of Embodiment 1.

FIG. 13 is a block diagram showing the configuration of a sound signal processing device according to Variation 6 of Embodiment 1.

FIG. 14 is a flowchart showing the processing procedure of the sound signal processing device according to Variation 6 of Embodiment 1.

FIG. 15 is a block diagram showing the configuration of a sound signal processing device according to Variation 7 of Embodiment 1.

FIG. 16 is a flowchart showing the processing procedure of the sound signal processing device according to Variation 7 of Embodiment 1.

FIG. 17 is a block diagram showing the configuration of a sound signal processing device according to Embodiment 2.

FIG. 18 is a flowchart showing the processing procedure of the sound signal processing device according to Embodiment 2.

### DESCRIPTION OF EMBODIMENTS

### (Circumstances Leading to the Present Disclosure)

Conventionally, patients with Alzheimer's dementia are known to accumulate a protein called amyloid β that is generated in the brain and accumulates without being excreted. Accumulated amyloid β destroys brain cells that are responsible for memory. This makes dementia patients more likely to forget things.

Here, it has been known that by exciting gamma waves in the brain with light, sound or the like, the production amount of amyloid β decreases, and microglia start to take in amyloid β, so the amount of amyloid β accumulated decreases. PTL 1 described above discloses a device that treats, prevents, or alleviates (hereinafter also referred to as “improves”) symptoms of dementia or Alzheimer's disease (hereinafter also simply referred to as “dementia and the like”).

Gamma waves are approximately 30 Hz to 90 Hz. If gamma waves are to be excited in the brain by sound, it is conceivable to make the subject listen to sounds with a frequency of about 30 Hz to 90 Hz, for example. However, there is a problem in that sounds with a frequency of about 30 Hz to 90 Hz tend to be unpleasant for many people.

As such, in view of these problems, the present inventors provide a sound signal processing method and the like that can suitably output sound in a specific frequency band to a target.

Hereinafter, each of the embodiments and variations will be specifically described with reference to the drawings. It should be noted that each of the embodiments and variations described below shows a comprehensive or specific example. The numerical values, shapes, materials, components, arrangement positions and connection forms of components, steps, order of the steps, etc. shown in each of the embodiments and variations below are mere examples, and are not intended to limit the present disclosure. In addition, among the components in each of the embodiments and variations below, those not recited in any one of the independent claims are described as optional components.

It should be noted that each figure is a schematic diagram and is not necessarily exactly illustrated. In addition, in each figure, substantially the same configurations are denoted by the same reference numerals, and overlapping explanations may be omitted or simplified.

### Embodiment 1

### [Configuration]

First, the configuration of the sound signal processing device according to Embodiment 1 will be described.

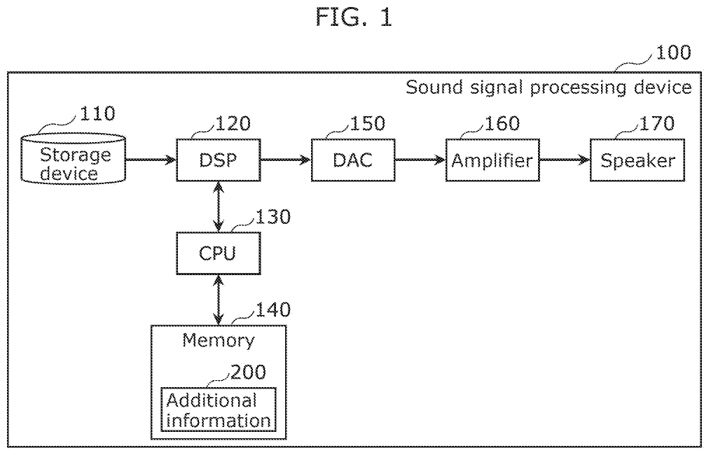

FIG. 1 is a block diagram showing the configuration of sound signal processing device 100 according to Embodiment 1.

Sound signal processing device 100 is a device (playback system) that outputs (plays) a sound signal based on sound content (sound information) such as music stored in storage device 110. Sound signal processing device 100 is, for example, a portable audio device having an earphone type device, a stationary audio device, or the like. It should be noted that sound signal processing device 100 only needs to be able to process and output sound signals as described later, and may be, for example, a personal computer, a smartphone, a tablet terminal, and the like each having a speaker.

It should be noted that sound signal processing device 100 may not include a speaker, but may have a configuration in which the speaker is externally attached. For example, sound signal processing device 100 may be configured to output an analog sound signal output from amplifier 160 to an external device such as a speaker or an earphone.

Specifically, sound signal processing device 100 includes storage device 110, DSP 120, CPU 130, memory 140, DAC 150, amplifier 160, and speaker 170.

Storage device 110 is a storage that stores sound content such as music content. Specifically, the sound content stored in storage device 110 is an example of first content, and the signal based on the sound content (sound source signal to be described later) is an example of the first sound signal. Storage device 110 is realized by, for example, a hard disk drive (HDD), a flash memory, or the like.

DSP 120 is a processor (Digital Signal Processor) that executes various processes by executing a control program stored in memory 140. Specifically, DSP 120 reads out a sound signal (first sound signal) from storage device 110 and performs signal processing on the read-out sound signal. More specifically, in order to increase the sound pressure of a component in a specific frequency band (hereinafter referred to as a specific frequency band) of the sound signal, DSP 120 performs processing to increase the level (signal level) of the component. The specific frequency band is, for example, at least 10 Hz and at most 100 kHz. The specific frequency band may be at least 40 Hz and at most 100 Hz. Alternatively, the specific frequency band may be at least 60 Hz and at most 100 Hz. Alternatively, the specific frequency band may be 40 Hz±10 Hz (that is, at least 30 Hz and at most 50 Hz). In addition, the specific frequency band may be a range such as 30 Hz to 50 Hz, or may be a specific frequency such as 40 Hz.

CPU 130 is a processor (Central Processing Unit) that executes various processes by executing a control program stored in memory 140. For example, CPU 130 obtains additional information 200 from memory 140.

Memory 140 is memory that stores additional information 200. Memory 140 is realized by, for example, a semiconductor memory or the like.

It should be noted that memory 140 may store a control program executed by DSP 120 and CPU 130. In addition, memory 140 may store information (threshold information) indicating thresholds and the like necessary for processing executed by DSP 120 and CPU 130.

Additional information 200 is sound content that includes a specific frequency band. Specifically, additional information 200 is sound content that includes only a specific frequency band. For example, if the sound signal based on additional information 200 is a signal in which a specific frequency band is a single frequency such as 40 Hz or the like, it is a signal containing a sine wave with the single frequency. Alternatively, for example, if the sound signal based on additional information 200 is a signal in which a specific frequency has a bandwidth such as 40 Hz±10 Hz or the like, it is a signal containing sine waves with a plurality of frequencies included in the range of the band. It should be noted that additional information 200 is an example of the second content, and the additional signal based on additional information 200 is an example of the second sound signal.

CPU 130 outputs a sound signal (second sound signal) based on obtained additional information 200 to DSP 120.

DSP 120 outputs a signal (superimposed signal) in which the first sound signal and the second sound signal are superimposed to DAC 150.

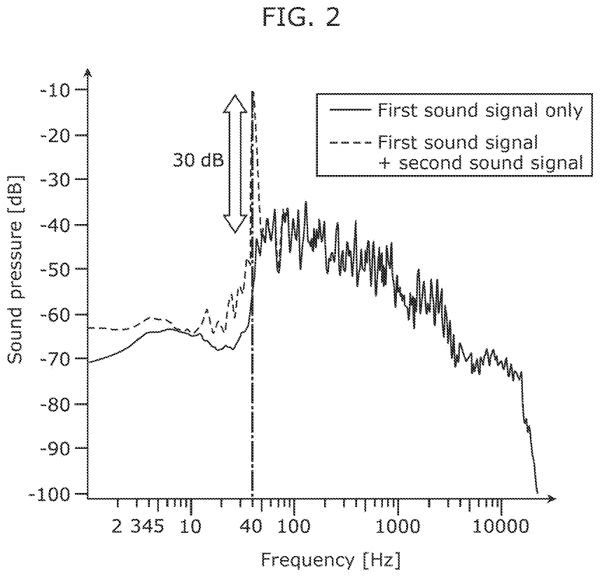

FIG. 2 is a diagram showing a first sound signal and a sound signal in which a second sound signal is superimposed on the first sound signal. Specifically, FIG. 2 is a diagram showing a graph obtained by Fourier transforming a first sound signal (first power spectrum) and a graph obtained by Fourier transforming a sound signal in which a second sound signal is superimposed on the first sound signal (second power spectrum). The horizontal axis of the graph shown in FIG. 2 indicates frequency (unit: Hz), and the vertical axis thereof indicates sound pressure (unit: dB). In addition, in FIG. 2, the first power spectrum is shown by a solid line, and the second power spectrum is shown by a broken line.

As shown by the broken line in FIG. 2, it can be seen that in the second power spectrum, for example, the sound pressure between 30 Hz and 50 Hz, particularly the sound pressure at 40 Hz, has increased. In this way, DSP 120 generates a signal in which the signal in the specific frequency band is enhanced by superimposing the first sound signal and the second sound signal. Specifically, DSP 120 adjusts the signal level of the second sound signal corresponding to the second content, which includes components in the specific frequency band, according to the signal level of the specific frequency band in the first sound signal corresponding to the first content, and superimposes and outputs the first sound signal and the adjusted second sound signal. For example, the second sound signal may be a signal that includes components other than those in the specific frequency band, or may be a signal that includes only components in the specific frequency band. Alternatively, for example, the second sound signal may be a signal that includes only a component of a specific frequency band and a component of overtones of the specific frequency band, which will be described later.

The method by which DSP 120 adjusts the signal level of the second sound signal is not particularly limited. For example, a method of calculating the envelope (envelope value) of the first sound signal and adjusting the signal of the second sound signal based on the calculated envelope is exemplified. In addition, for example, a method of calculating the signal level in a specific frequency band of the first sound signal by performing Fourier transform (that is, short-time Fourier transform) on the first sound signal over a short predetermined time, and adjusting the signal level of the second sound signal according to the calculated signal level, and the like is exemplified. A specific adjustment method will be described later.

DAC 150 is a converter (Digital Analog Converter) that converts the signal obtained from DSP 120 from a digital signal to an analog signal. DAC 150 outputs an analog signal to amplifier 160.

Amplifier 160 is an amplifier that amplifies an analog signal. Amplifier 160 outputs the amplified analog signal to speaker 170.

Speaker 170 outputs sound based on the analog signal obtained from amplifier 160. Speaker 170 may be a speaker shaped to be worn in the ear canal, or it may be a stationary speaker. In addition, speaker 170 may be a speaker that emits sound waves toward the eardrum, or may be a bone conduction speaker.

It should be noted that the processing of DSP 120 and the processing of CPU 130 may be executed by either DSP 120 or CPU 130. DSP 120 and CPU 130 may be realized by one processor. DSP 120 and CPU 130 may be realized by one microcontroller (microcomputer) or may be realized by a plurality of microcomputers. DSP 120, CPU 130, memory 140, and DAC 150 may be realized by one system-on-a-chip (SoC) or by a plurality of SoCs. DSP 120, CPU 130, memory 140, and DAC 150 may be realized by any combination of the above configurations.

### [Processing Procedure]

Subsequently, the processing procedure of sound signal processing device 100 will be explained.

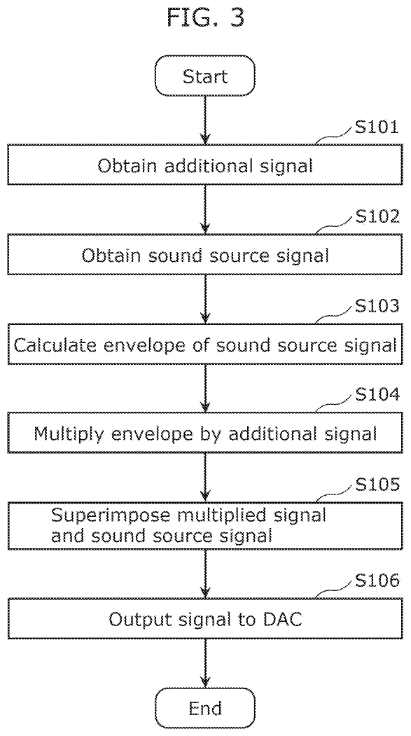

FIG. 3 is a flowchart showing the processing procedure of sound signal processing device 100 according to Embodiment 1. Specifically, FIG. 3 is a flowchart showing the processing procedure of DSP 120.

First, DSP 120 obtains, from CPU 130, an additional signal that is a sound signal based on additional information 200 that CPU 130 obtained from memory 140 (S101).

Next, DSP 120 reads out the sound content from storage device 110 to obtain a sound source signal that is a sound signal based on the sound content (S102).

Next, DSP 120 uses Hilbert transform to calculate the envelope of the sound source signal (S103).

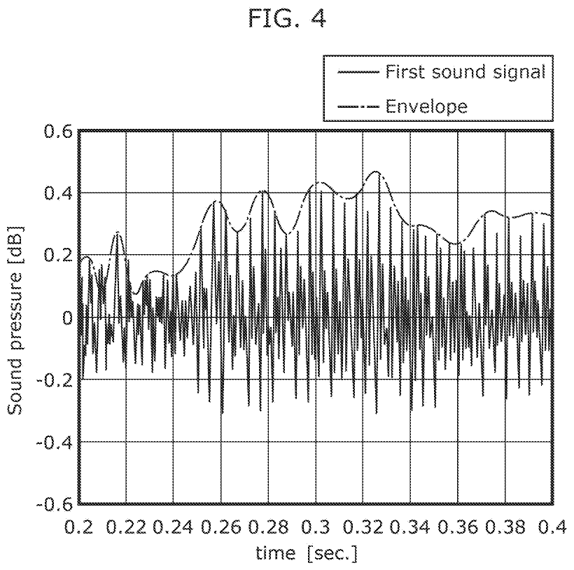

FIG. 4 is a diagram for explaining the calculation result of the envelope calculated from the first sound signal. Specifically, FIG. 4 is a graph showing the temporal change in the signal level of a first sound signal and the envelope calculated from the first sound signal. The horizontal axis of the graph shown in FIG. 4 indicates time (unit: seconds), and the vertical axis thereof indicates sound pressure (unit: dB). It should be noted that in FIG. 4, the first sound signal is shown by a solid line, and the envelope is shown by a chain line.

The envelope calculated from the first sound signal is a line (n-th order function/n: natural number) provided so as to be in contact with a plurality of local maximum values of the first sound signal. It should be noted that the envelope calculated from the first sound signal may be provided so as to be in contact with all the local maximum values of the first sound signal, or it may be provided so as to be in contact with any two or more local maximum values instead of all the local maximum values.

Referring again to FIG. 3, after step S103, DSP 120 multiplies the calculated envelope by the additional signal (S104). Specifically, DSP 120 generates a signal (multiplied signal) by multiplying the calculated envelope and the additional signal.

For example, the additional signal is a signal in which the signal level in a specific frequency band is m (m>0) and the signal level in other frequency bands other than the specific frequency band is zero. m may be set arbitrarily and is not particularly limited. For example, m=1. When such an additional signal is multiplied by the envelope, the envelope in a specific frequency band (more specifically, the signal level of the envelope) is multiplied by m, and the envelopes in other frequency bands become zero. That is, in this case, in the multiplied signal, the signal level in the specific frequency band is m times the envelope, and the signal level in other frequency bands becomes zero. In this way, the envelope has a value that corresponds to the signal level of the sound source signal, so the multiplied signal has a value that corresponds to the signal level of the sound source signal.

Next, DSP 120 superimposes (adds) the generated multiplied signal and the sound source signal (S105). Specifically, DSP 120 generates a signal (superimposed signal) by superimposing the generated multiplied signal and the sound source signal. Accordingly, the superimposed signal becomes a signal in which the signal level of the specific frequency band in the sound source signal is corrected according to the signal level of the sound source signal. In other words, DSP 120 can add, to the sound source signal, a signal with a signal level corresponding to the signal level of the sound source signal.

Next, DSP 120 outputs (transmits) the generated signal (superimposed signal) to DAC 150 (S106).

The superimposed signal is transmitted from DAC 150 to speaker 170 via amplifier 160. Speaker 170 outputs sound based on the superimposed signal.

### [Variations]

Hereinafter, each variation will be explained. It should be noted that the following description will focus on the differences from Embodiment 1 described above or each variation described later.

### <Variation 1>

The sound signal processing device according to Variation 1 has the same configuration as sound signal processing device 100 shown in FIG. 1, but the processing procedure is different. Specifically, DSP 120 according to Variation 1 sets the signal level of the second sound signal to a predetermined level when the signal level of the first sound signal is lower than or equal to the threshold. Accordingly, even when the signal level of the first sound signal is too low, the second sound signal of a predetermined level is superimposed, so that the signal level of the specific frequency band can be prevented from falling too much.

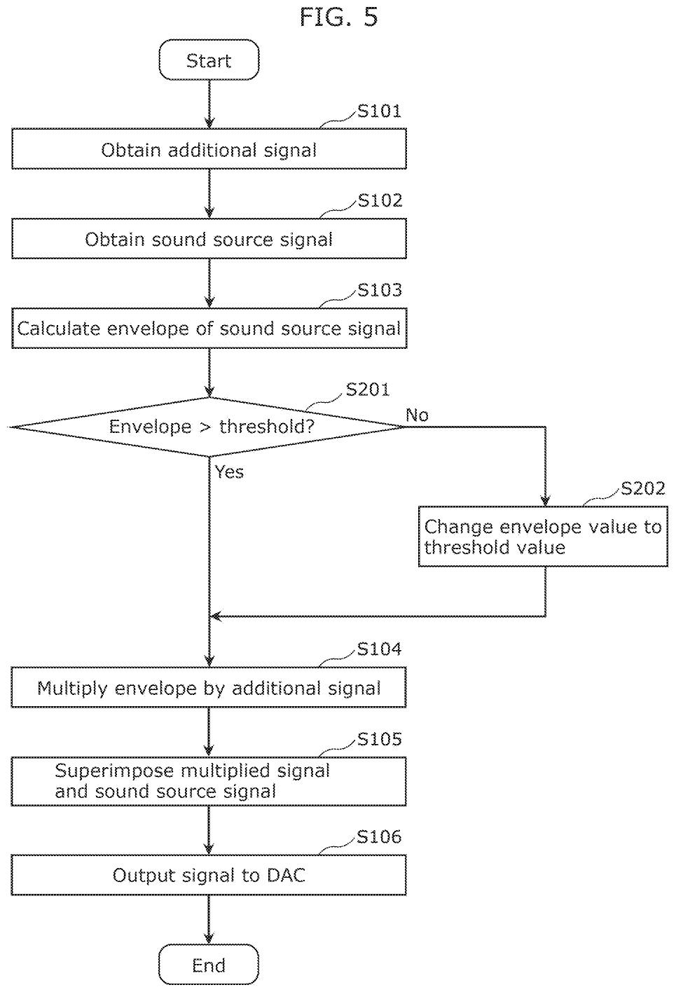

FIG. 5 is a flowchart showing the processing procedure of the sound signal processing device according to Variation 1 of Embodiment 1. Specifically, FIG. 5 is a flowchart showing the processing procedure of DSP 120 included in the sound signal processing device according to Variation 1.

First, DSP 120 obtains, from CPU 130, an additional signal that is a sound signal based on additional information 200 that CPU 130 obtained from memory 140 (S101).

Next, DSP 120 reads out a sound content from storage device 110 to obtain a sound source signal that is a sound signal based on the sound content (S102).

Next, DSP 120 uses Hilbert transform to calculate the envelope of the sound source signal (S103).

Next, DSP 120 determines whether the calculated envelope is larger than a threshold (first threshold) (S201). The first threshold may be arbitrarily determined in advance and is not particularly limited. First threshold information indicating the first threshold is stored in memory 140 in advance, for example. DSP120 obtains, from CPU 130, the first threshold information which CPU 130 obtained from memory 140, for example.

When DSP 120 determines that the calculated envelope is lower than or equal to the threshold (No in S201), DSP 120 changes the value of the envelope to the value of the threshold (S202). Specifically, DSP 120 changes each value that makes up the envelope so that, among the values that make up the envelope, the values that are higher than the threshold remain as they are, and the values that are lower than or equal to the threshold become the value of the threshold.

This prevents the envelope value from becoming too small.

If Yes in step S201 or after step S202, DSP 120 generates a multiplied signal by multiplying the calculated envelope and the additional signal (S104).

Next, DSP 120 generates a signal (superimposed signal) by superimposing the generated multiplied signal and the sound source signal (S105).

Next, DSP 120 outputs the generated signal (superimposed signal) to DAC 150 (S106).

The superimposed signal is transmitted from DAC 150 to speaker 170 via amplifier 160. Speaker 170 outputs sound based on the superimposed signal.

As described above, in the adjustment of the signal level of the second sound signal, DSP 120 according to Variation 1 raises the signal level of the second sound signal by a predetermined ratio to the signal level of the specified frequency band in the first sound signal if the signal level of the specified frequency band in the first sound signal is higher than the threshold value, and sets the signal level of the second sound signal at the predetermined level if the signal level of the first sound signal is lower than the threshold value.

It should be noted that the predetermined ratio and the predetermined level may be arbitrarily determined in advance and are not particularly limited. In this example, the predetermined ratio is determined by the envelope. For example, in step S104, a predetermined value that was determined in advance may be further multiplied and/or added to the envelope.

### <Variation 2>

The sound signal processing device according to Variation 2 has the same configuration as sound signal processing device 100 shown in FIG. 1, but the processing procedure is different. Specifically, DSP 120 according to Variation 2 generates a multiplied signal by multiplying a signal (corresponding signal) obtained from the calculation result of FFT of the sound source signal instead of the envelope by the additional signal. This also allows a signal having a signal level corresponding to the sound source signal to be superimposed on the sound source signal, as in the case of using the envelope.

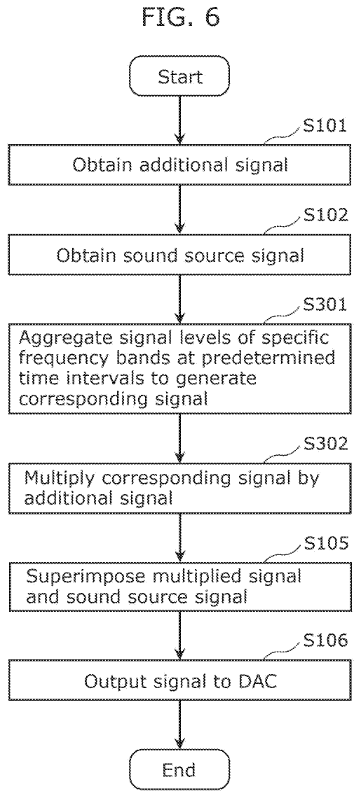

FIG. 6 is a flowchart showing the processing procedure of the sound signal processing device according to Variation 2 of Embodiment 1. Specifically, FIG. 6 is a flowchart showing the processing procedure of DSP 120 included in the sound signal processing device according to Variation 2.

First, DSP 120 obtains, from CPU 130, an additional signal that is a sound signal based on additional information 200 that CPU 130 obtained from memory 140 (S101).

Next, DSP 120 reads out a sound content from storage device 110 to obtain a sound source signal that is a sound signal based on the sound content (S102).

Next, DSP 120 aggregates the signal levels of a specific frequency band (in other words, the frequency band corresponding to the additional signal) at predetermined time intervals, and generates a corresponding signal based on the aggregated signal levels (S301).

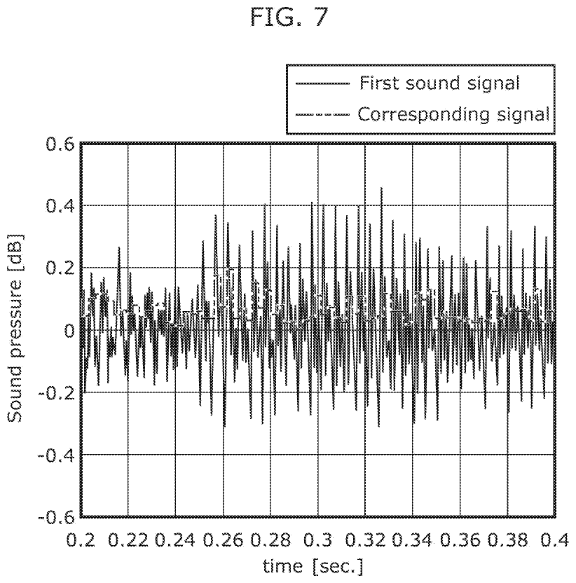

FIG. 7 is a diagram for explaining a signal (corresponding signal) obtained from the calculation result of performing FFT on the first sound signal. Specifically, FIG. 7 is a graph showing a temporal change in the signal level of the first sound signal and a signal obtained from the calculation result of performing FFT on the first sound signal. The horizontal axis of the graph shown in FIG. 7 indicates time (unit: seconds), and the vertical axis thereof indicates sound pressure (unit: dB). It should be noted that in FIG. 7, the first sound signal is shown by a solid line, and the signal obtained from the FFT calculation result is shown by a two-dot chain line.

For example, DSP 120 performs FFT on the first sound signal every predetermined time interval. Next, DSP 120 determines the signal level of the specific frequency band in the calculation result of performing FFT on the first sound signal as the signal level for each predetermined time interval. Next, DSP 120 generates a signal at the determined signal level for each predetermined time interval. Accordingly, DSP 120 generates a corresponding signal whose signal level is constant within a predetermined time interval, such as the signal shown by the two-dot chain line in FIG. 7.

It should be noted that the predetermined time may be arbitrarily determined in advance and is not particularly limited. The predetermined time is, for example, on the order of several milliseconds. In the example shown in FIG. 7, the predetermined time is 2.5 milliseconds. Time information indicating a predetermined time is stored in advance in memory 140, for example. DSP120 obtains, from CPU 130, the time information which CPU 130 obtained from memory 140, for example.

In addition, the sound signal processing device according to Variation 2 may include a timer such as a real time clock (RTC) to measure time.

Referring again to FIG. 6, after step S301, DSP 120 generates a multiplied signal by multiplying the generated corresponding signal by the additional signal (S302).

Next, DSP 120 generates a signal (superimposed signal) by superimposing the generated multiplied signal and the sound source signal (S105).

Next, DSP 120 outputs the generated signal (superimposed signal) to DAC 150 (S106).

The superimposed signal is transmitted from DAC 150 to speaker 170 via amplifier 160. Speaker 170 outputs sound based on the superimposed signal.

### <Variation 3>

The sound signal processing device according to Variation 3 has the same configuration as sound signal processing device 100 shown in FIG. 1, but the processing procedure is different. Specifically, DSP 120 according to Variation 3 controls to increase the signal level of the frequency band of the overtone of the specific frequency band of the first sound signal. It is known that overtones in a specific frequency band are also effective in improving dementia and the like, similar to sounds in a specific frequency band. For that reason, sounds that are more effective in improving dementia and the like are output.

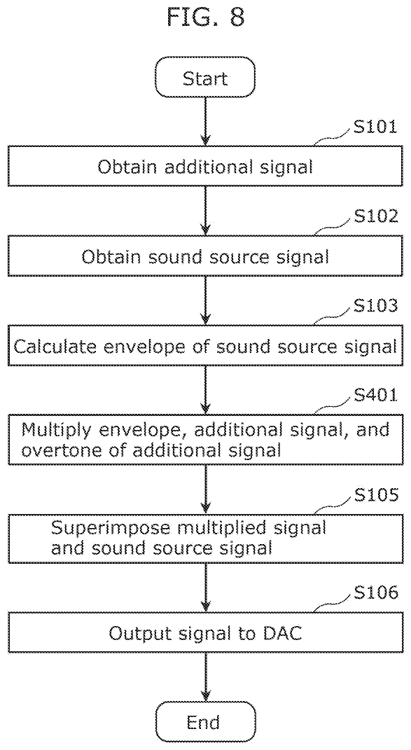

FIG. 8 is a flowchart showing the processing procedure of the sound signal processing device according to Variation 3 of Embodiment 1. Specifically, FIG. 8 is a flowchart showing the processing procedure of DSP 120 included in the sound signal processing device according to Variation 3.

First, DSP 120 obtains, from CPU 130, an additional signal that is a sound signal based on additional information 200 that CPU 130 obtained from memory 140 (S101).

Next, DSP 120 reads out the sound content from storage device 110 to obtain a sound source signal that is a sound signal based on the sound content (S102).

Next, DSP 120 uses Hilbert transform to calculate the envelope of the sound source signal (S103).

Next, DSP 120 generates a multiplied signal by multiplying the calculated envelope, the additional signal, and the overtone (overtone signal) of the additional signal (S401).

For example, if the specific frequency band is 40 Hz±10 Hz, DSP 120 generates an overtone signal of 80 Hz±20 Hz. It should be noted that the overtone of the specific frequency band may not only have a frequency twice as high as the specific frequency band, but also a frequency p times (p: natural number) as high as the specific frequency band. In addition, the overtone signal may include only a signal of one frequency, or may include signals of a plurality of frequencies. For example, the overtone signal may include a signal with a frequency twice as high as the specific frequency band and a signal with a frequency three times as high as the specific frequency band. Overtone information indicating overtone signals may be stored in memory 140 in advance. In this case, DSP 120 obtains, from CPU 130, the overtone information that CPU 130 obtained from memory 140, for example.

Next, DSP 120 generates a signal (superimposed signal) by superimposing the generated multiplied signal and the sound source signal (S105).

Next, DSP 120 outputs the generated signal (superimposed signal) to DAC 150 (S106).

The superimposed signal is transmitted from DAC 150 to speaker 170 via amplifier 160. Speaker 170 outputs sound based on the superimposed signal.

As described above, in adjusting the signal level of the second sound signal, for example, DSP 120 according to Variation 4 further controls (adjusts) to increase the signal level of the frequency band of the overtone of the specific frequency band.

### <Variation 4>

In Variation 4, the signal level of the additional signal is controlled based on the control information. According to this, for example, by receiving control information from the user, it is possible to output sound in a specific frequency band at a sound volume desired by the user.

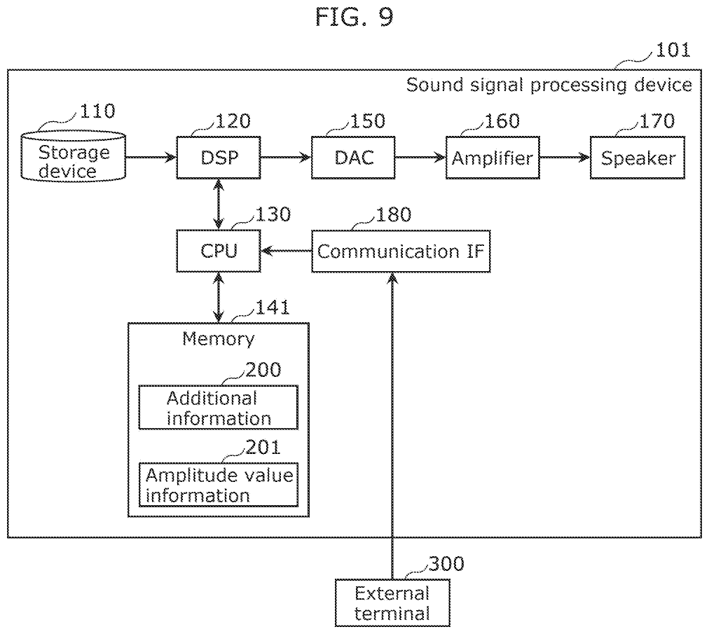

FIG. 9 is a block diagram showing the configuration of sound signal processing device 101 according to Variation 4 of Embodiment 1.

Sound signal processing device 101 includes storage device 110, DSP 120, CPU 130, memory 141, DAC 150, amplifier 160, speaker 170, and communication IF 180.

Memory 141 is memory that stores additional information 200 and amplitude value information 201. Memory 141 is realized by, for example, a semiconductor memory or the like.

Amplitude value information 201 is an example of control information, and is information for determining the sound pressure of additional information 200 (more specifically, the signal level of the additional signal based on additional information 200). Amplitude value information 201 is information indicating processing content such as “ON”, “OFF”, “UP”, or “DOWN”.

For example, when amplitude value information 201 indicates “ON”, DSP 120 multiplies the signal level of the additional signal based on additional information 200 by 1.0, adds it to the sound source signal stored in storage device 110, and outputs it to DAC 150.

Alternatively, for example, when amplitude value information 201 indicates “OFF”, DSP 120 multiplies the signal level of the additional signal based on additional information 200 by 0, adds it to the sound source signal stored in storage device 110, and outputs it to DAC 150. That is, in this case, for example, DSP 120 outputs the additional signal based on additional information 200 to DAC 150 without adding it to the sound source signal.

Alternatively, for example, when amplitude value information 201 indicates “UP”, DSP 120 multiplies the signal level of the additional signal based on additional information 200 by 1.1, adds it to the sound source signal stored in storage device 110, and outputs it to DAC 150.

Alternatively, for example, when amplitude value information 201 indicates “DOWN”, DSP 120 multiplies the signal level of the additional signal based on additional information 200 by 0.9, adds it to the sound source signal stored in storage device 110, and outputs it to DAC 150.

In these ways, for example, DSP 120 obtains control information indicating the signal level of a specific frequency band, and in adjusting the signal level of the second sound signal, the signal level of the specific frequency band is adjusted based on the control information. Specifically, for example, DSP 120 switches the additional signal on/off (that is, whether the additional signal is superimposed on the sound source signal) or switches the signal level based on amplitude value information 201.

It should be noted that amplitude value information 201 may be information indicating a numerical value.

For example, when amplitude value information 201 indicates “1.0”, DSP 120 multiplies the signal level of the additional signal based on additional information 200 by 1.0, adds it to the sound source signal stored in storage device 110, and outputs it to DAC 150.

Alternatively, for example, when amplitude value information 201 indicates “0.0”, DSP 120 multiplies the signal level of the additional signal based on additional information 200 by 0, adds it to the sound source signal stored in storage device 110, and outputs it to DAC 150. That is, in this case, for example, DSP 120 outputs the additional signal based on additional information 200 to DAC 150 without adding it to the sound source signal.

Alternatively, for example, when amplitude value information 201 indicates “1.1”, DSP 120 multiplies the signal level of the additional signal based on additional information 200 by 1.1, adds it to the sound source signal stored in storage device 110, and outputs it to DAC 150.

Alternatively, for example, when amplitude value information 201 indicates “0.9”, DSP 120 multiplies the signal level of the additional signal based on additional information 200 by 0.9, adds it to the sound source signal stored in storage device 110, and outputs it to DAC 150.

In these ways, amplitude value information 201 only needs to be any information that indicates how to set the signal level of the additional signal.

Amplitude value information 201 is obtained from external terminal 300 via communication IF 180, for example.

Communication IF 180 is a communication interface (IF) for communication between sound signal processing device 101 and external terminal 300. For example, when sound signal processing device 101 and external terminal 300 communicate wirelessly, communication IF 180 is realized by an antenna and a wireless communication circuit. Alternatively, communication IF 180 is realized by, for example, a connector to which a communication line is connected when sound signal processing device 101 and external terminal 300 communicate by wire.

It should be noted that the communication standard adopted for communication may be a communication standard such as Bluetooth (registered trademark) or Bluetooth (registered trademark) Low Energy (BLE), or may be an original communication standard, and is not particularly limited.

External terminal 300 is a communication terminal operated by a user. External terminal 300 is a terminal such as a console, a smartphone, or the like. A user transmits amplitude value information 201 to sound signal processing device 101 by operating external terminal 300. Accordingly, by operating external terminal 300, the user can switch on/off the additional signal based on additional information 200 or change the signal level. For example, CPU 130 causes memory 141 to store amplitude value information 201 obtained from external terminal 300 via communication IF 180. For example, each time CPU 130 obtains amplitude value information 201 from external terminal 300 via communication IF 180, CPU 130 updates amplitude value information 201 stored in memory 141.

It should be noted that memory 141 may store a control program executed by DSP 120 and CPU 130. In addition, memory 141 may store information (threshold information) indicating thresholds and the like necessary for processing executed by DSP 120 and CPU 130.

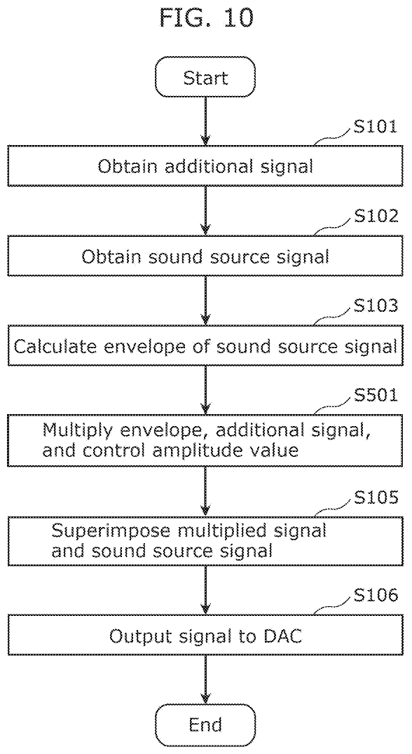

FIG. 10 is a flowchart showing the processing procedure of sound signal processing device 101 according to Variation 4 of Embodiment 1. Specifically, FIG. 10 is a flowchart showing the processing procedure of DSP 120 included in sound signal processing device 101 according to Variation 4.

First, DSP 120 obtains, from CPU 130, an additional signal that is a sound signal based on additional information 200 that CPU 130 obtained from memory 141 (S101). In addition, for example, DSP 120 obtains, from CPU 130, amplitude value information 201 that CPU 130 obtained from memory 141.

Next, DSP 120 reads the sound content from storage device 110 to obtain a sound source signal that is a sound signal based on the sound content (S102).

Next, DSP 120 uses Hilbert transform to calculate the envelope of the sound source signal (S103).

Next, DSP 120 generates a multiplied signal by multiplying the calculated envelope, the additional signal, and, for example, a numerical value indicated by amplitude value information 201 (S501).

Next, DSP 120 generates a signal (superimposed signal) by superimposing the generated multiplied signal and the sound source signal (S105).

Next, DSP 120 outputs the generated signal (superimposed signal) to DAC 150 (S106).

The superimposed signal is transmitted from DAC 150 to speaker 170 via amplifier 160. Speaker 170 outputs sound based on the superimposed signal.

As described above, for example, DSP 120 obtains control information (for example, amplitude value information 201) indicating the signal level of a specific frequency band, and adjusts (controls) the signal level of the second sound signal based on the control information.

### <Variation 5>

In Variation 5, the signal level of the additional signal is controlled based on the user's biological information. According to this, for example, it is possible to output sound in a specific frequency band at a sound volume that corresponds to the user's comfort level.

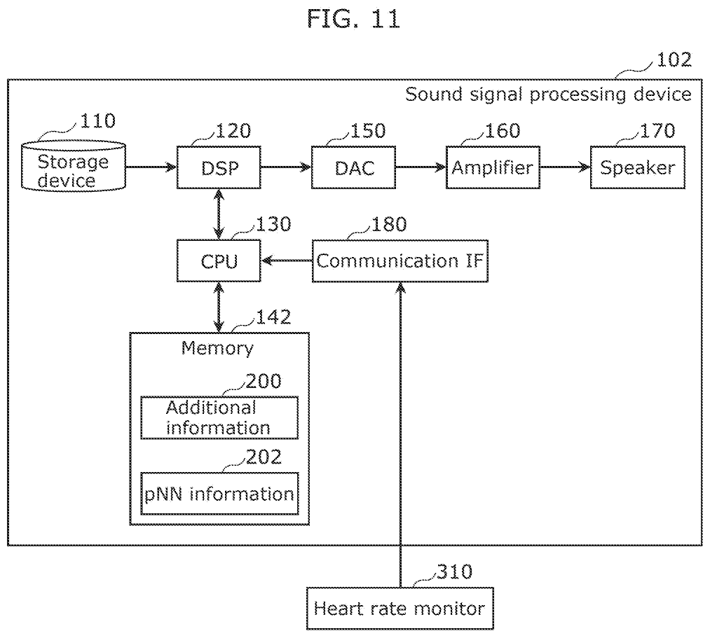

FIG. 11 is a block diagram showing the configuration of sound signal processing device 102 according to Variation 5 of Embodiment 1.

Sound signal processing device 102 includes storage device 110, DSP 120, CPU 130, memory 142, DAC 150, amplifier 160, speaker 170, and communication IF 180.

Memory 142 is memory that stores additional information 200 and pNN information 202. Memory 142 is realized by, for example, a semiconductor memory or the like.

pNN information 202 is information for determining the sound pressure of additional information 200. Specifically, pNN information 202 is an example of biological information, and is information indicating a pNN50 value. The pNN50 value is the percentage of heartbeats in which the difference between consecutive adjacent RR intervals exceeds 50 ms.

For example, CPU 130 repeatedly obtains pNN information 202 from heart rate monitor 310 via communication IF 180 and stores it in memory 142. Accordingly, memory 142 stores pNN information 202 indicating the temporal change in the user's pNN50 value.

Heart rate monitor 310 is a device that measures the user's heart rate, calculates a pNN50 value, and repeatedly transmits pNN information 202 indicating the calculation result to sound signal processing device 102.

It should be noted that the time interval at which heart rate monitor 310 repeatedly transmits pNN information 202 may be arbitrarily determined in advance and is not particularly limited.

DSP 120 switches the signal level of the additional signal based on pNN information 202. For example, when the pNN50 value indicated by pNN information 202 decreases, DSP 120 reduces the signal level of the additional signal.

It is known that the pNN50 value is a value that reflects the user's comfort/discomfort (whether the user is comfortable). For example, if the pNN50 value decreases when some stimulus is given to the user, there is a high possibility that the user is feeling uncomfortable. As such, DSP 120 obtains the user's biological information, for example, and controls (adjusts) the signal level of a specific frequency band based on the biological information in adjusting the signal level of the second sound signal. Specifically, for example, when the pNN50 value indicated by pNN information 202 decreases, DSP 120 reduces the signal level of the additional signal.

It should be noted that, for example, when the pNN50 value indicated by pNN information 202 increases, DSP 120 may increase the signal level of the additional signal.

In addition, for example, CPU 130 may obtain information indicating the user's heartbeat, such as an electrocardiogram, from heart rate monitor 310 via communication IF 180, calculate the user's pNN50 value based on the obtained information, and store information indicating the calculation result in memory 142 as pNN information 202.

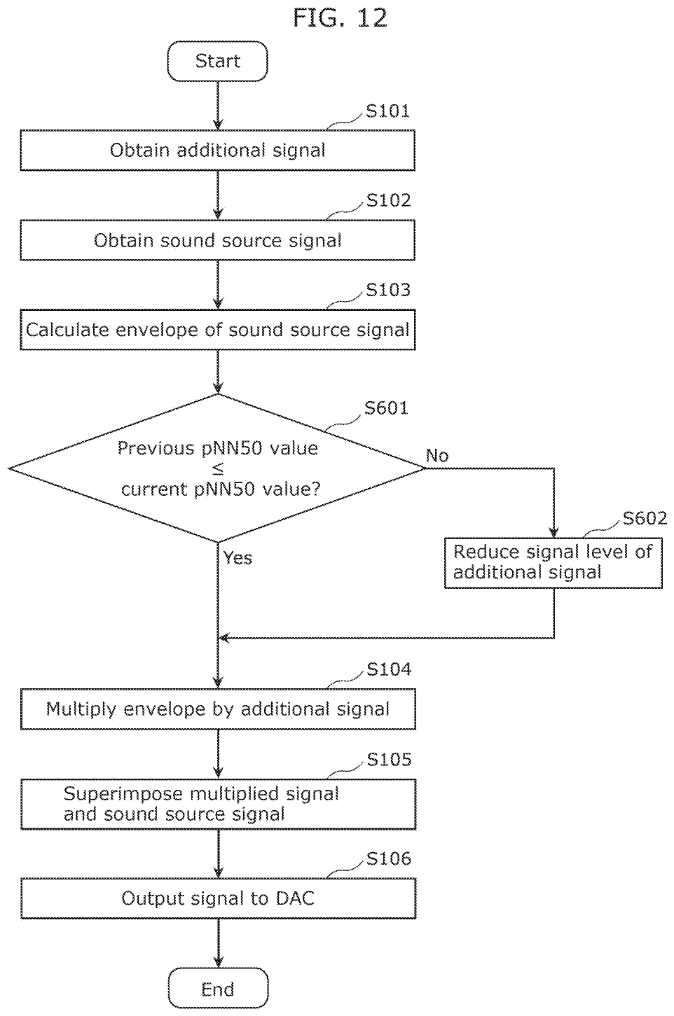

FIG. 12 is a flowchart showing the processing procedure of sound signal processing device 102 according to Variation 5 of Embodiment 1. Specifically, FIG. 12 is a flowchart showing the processing procedure of DSP 120 included in sound signal processing device 102 according to Variation 5.

First, DSP 120 obtains, from CPU 130, an additional signal that is a sound signal based on additional information 200 that CPU 130 obtained from memory 142 (S101).

Next, DSP 120 reads out the sound content from storage device 110 to obtain a sound source signal that is a sound signal based on the sound content (S102).

Next, DSP 120 uses Hilbert transform to calculate the envelope of the sound source signal (S103).

DSP 120 obtains, from CPU 130, pNN information 202 that CPU 130 obtained from memory 142, and determines whether the current pNN50 value is greater than or equal to the previous pNN50 value based on obtained pNN information 202 (S601). Specifically, DSP 120 determines whether the latest pNN value is greater than or equal to the pNN50 value immediately before the pNN value.

If DSP 120 determines that the current pNN50 value is less than the previous pNN50 value (No in S601), it reduces the signal level of the additional signal based on additional information 200 (S602). The signal level that DSP 120 reduces may be arbitrarily determined in advance. In addition, the signal level that DSP 120 reduces may be determined based on the difference between the previous pNN50 value and the current pNN50 value. For example, DSP 120 may reduce the signal level of the additional signal to a greater extent as the difference is larger.

If Yes in step S601 or after step S602, DSP 120 generates a multiplied signal by multiplying the calculated envelope by the additional signal (S104).

Next, DSP 120 generates a signal (superimposed signal) by multiplying the generated multiplied signal and the sound source signal (S105).

Next, DSP 120 outputs the generated signal (superimposed signal) to DAC 150 (S106).

The superimposed signal is transmitted from DAC 150 to speaker 170 via amplifier 160. Speaker 170 outputs sound based on the superimposed signal.

It should be noted that in this example, the process of changing the signal level of the additional signal is performed based on pNN information 202 obtained from heart rate monitor 310. The process of changing the signal level of the additional signal may be performed based on other biological information rather than pNN information 202. Biological information is information that indicates the degree to which the user feels comfortable. The other biological information may be, for example, information indicating the user's breathing rate, information indicating the user's body temperature, information indicating the user's sweat amount, information indicating the user's brain waves, or information indicating the user's facial expression (for example, image information). The process of changing the signal level of the additional signal may be performed based on the user's biological information. For example, DSP 120 reduces the level of the second sound signal when the biological information indicates that the user feels uncomfortable (specifically, indicates that the degree of comfort has decreased).

For example, CPU 130 may obtain the user's biological information from a device that obtains the user's biological information, such as a thermometer, an electroencephalogram, or a camera, via communication IF 180, and store it in memory 142. DSP 120 may adjust the signal level of the additional signal based on temporal changes in the user's biological information.

### <Variation 6>

In Variation 6, the sound output from speaker 170 is collected, and the signal level of the second sound signal is adjusted based on the collected sound. According to this, it is possible to output sound from speaker 170 at an appropriate sound volume according to the installation environment of speaker 170 and the like.

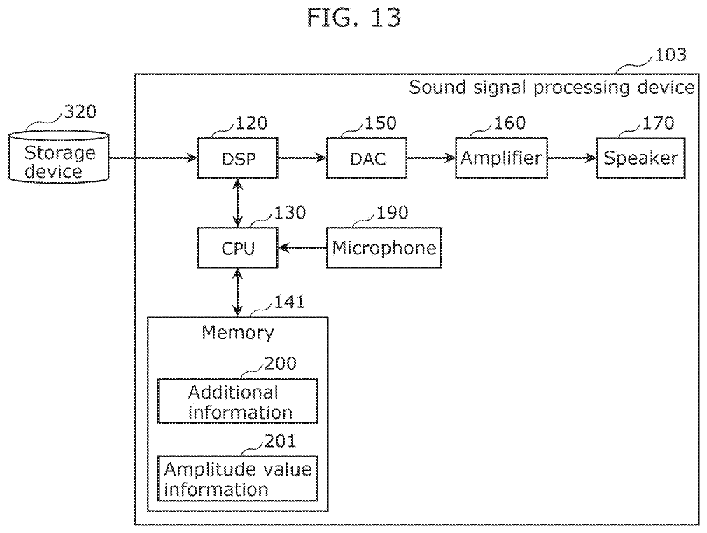

FIG. 13 is a block diagram showing the configuration of sound signal processing device 103 according to Variation 6 of Embodiment 1.

Sound signal processing device 103 includes DSP 120, CPU 130, memory 141, DAC 150, amplifier 160, speaker 170, and microphone 190.

In this way, for example, sound signal processing device 103 does not include storage device 110. In such a case, for example, sound signal processing device 103 obtains the sound source signal and the like from storage device 320 included in an external device such as a communicably connected server device. Of course, sound signal processing device 103 may include a communication IF for communicating with the server device or the like.

According to this, sound signal processing device 103 is realized without having large-sized components such as storage device 110, so that sound signal processing device 103 can be miniaturized, for example, and can be realized as an earphone, for example.

Microphone 190 is a microphone that collects the sound output from speaker 170 and outputs a sound signal (hereinafter also referred to as a microphone signal) based on the collected sound. Microphone 190 is a microphone such as a condenser microphone, a dynamic microphone, a micro electro mechanical systems (MEMS) microphone, or the like. For example, when sound signal processing device 103 is an earphone, microphone 190 is a so-called earphone microphone that is housed in the housing of the earphone.

Microphone 190 outputs a microphone signal based on the collected sound to CPU 130.

For example, CPU 130 updates amplitude value information 201 based on the microphone signal. In other words, CPU 130 obtains, from microphone 190, a microphone signal (output sound signal) that was detected by microphone 190 and is based on the sound (output sound) output from speaker 170.

DSP 120 adjusts the signal level of the second sound signal based on amplitude value information 201. That is, DSP 120 further controls (adjusts) the signal level of the specific frequency band in the second sound signal based on the output sound signal based on the output sound output from speaker 170.

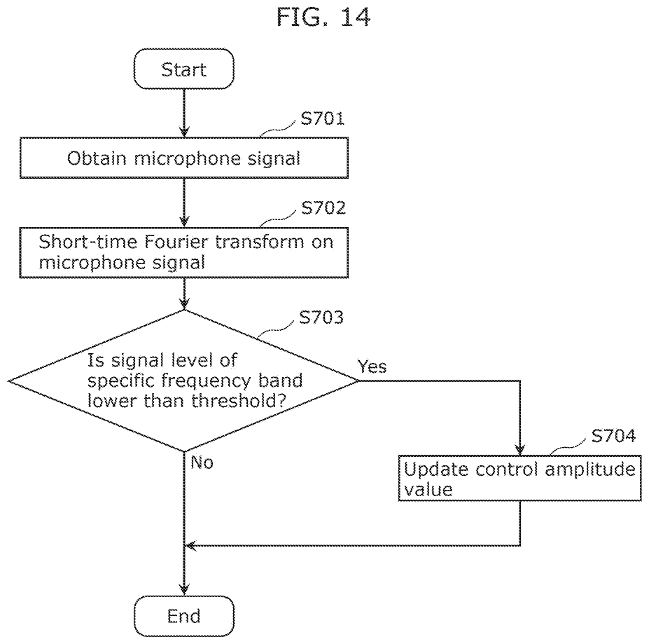

FIG. 14 is a flowchart showing the processing procedure of sound signal processing device 103 according to Variation 6 of Embodiment 1. Specifically, FIG. 14 is a flowchart showing the processing procedure for updating amplitude value information 201, which is executed by CPU 130 included in sound signal processing device 103 according to Variation 6.

First, CPU 130 obtains a microphone signal (output sound signal) from microphone 190 (S701).

Next, CPU 130 checks the signal level of the band (specific frequency band) corresponding to the additional signal, and performs short-time Fourier transform on the microphone signal (S702). The time for Fourier transformation may be arbitrarily determined and is not particularly limited.

CPU 130 determines whether the signal level of the specific frequency band in the microphone signal is lower than a predetermined threshold (second threshold) (S703). The second threshold information indicating the second threshold may be stored in memory 141 in advance, for example, and is not particularly limited. The second threshold information indicating the second threshold is stored in memory 140 in advance, for example. For example, DSP 120 obtains, from CPU 130, the second threshold information that CPU 130 obtained from memory 140.

It should be noted that the first threshold and the second threshold described above may be the same value or may be different values.

When CPU 130 determines that the signal level of the specific frequency band in the microphone signal is lower than the predetermined threshold (Yes in S703), CPU 130 updates amplitude value information 201 (more specifically, the control amplitude value indicated by amplitude value information 201) (S704).

Depending on the installation environment of speaker 170, speaker 170 may not output sound in a specific frequency band at the expected sound volume. As such, the sound actually output from speaker 170 is collected by microphone 190, and amplitude value information 201 is updated using a microphone signal based on the collected sound, that is, the sound pressure (sound volume) in the specific frequency band is controlled.

DSP 120 uses amplitude value information 201 updated as described above to perform the processing shown in FIG. 10, for example.

### <Variation 7>

There is a problem that when a subject is made to listen to a sound with a frequency of about 30 Hz to 90 Hz through both ears using earphones, for example, if there is a phase difference between the sound emitted from the speaker attached to the right ear and the sound emitted from the speaker attached to the left ear, it is difficult to obtain the effect of improving dementia and the like. As such, in Variation example 7, processing is performed to reduce the phase difference between the two sound signals (more specifically, to align the phases). This makes it easier to obtain the effect of improving dementia and the like.

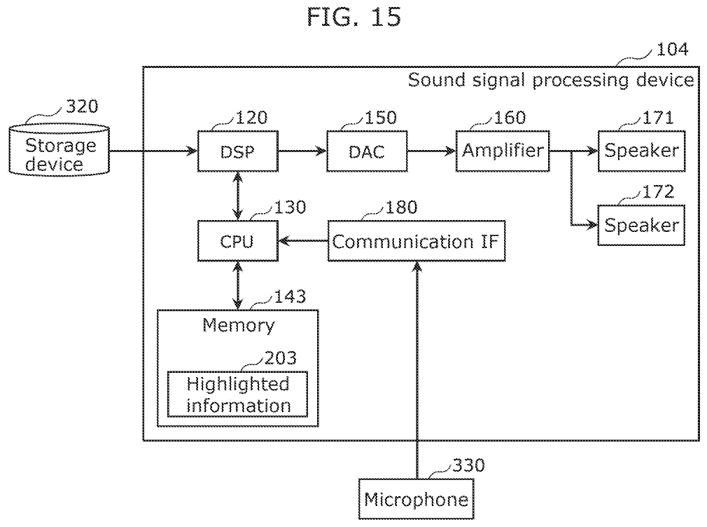

FIG. 15 is a block diagram showing the configuration of sound signal processing device 104 according to Variation 7 of Embodiment 1.

Sound signal processing device 104 includes DSP 120, CPU 130, memory 143, DAC 150, amplifier 160, speaker 171, speaker 172, and communication IF 180.

Memory 143 is memory that stores highlighted information 203. Memory 143 is realized by, for example, a semiconductor memory or the like.

It should be noted that memory 143 may store a control program executed by DSP 120 and CPU 130. In addition, memory 143 may store information (threshold information) indicating thresholds and the like necessary for processing executed by DSP 120 and CPU 130.

Highlighted information 203 is information for raising (enhancing) the signal level of a specific frequency band in the sound signal obtained by DSP 120. Highlighted information 203 includes, for example, information indicating a specific frequency band and information indicating a predetermined signal level. For example, DSP 120 performs processing to raise the signal level of a specific frequency band in an environmental sound signal (described later) to a predetermined level, based on highlighted information 203.

Speakers 171 and 172 each output sound based on the analog signal obtained from amplifier 160. Speakers 171 and 172 are, for example, a speaker shaped to be worn to an ear canal (that is, an earphone type speaker). For example, speaker 171 is an earphone worn in the left ear, and speaker 171 is an earphone worn in the right ear.

For example, storage device 320 stores the sound content of a sound signal output from an earphone worn to the left ear (hereinafter also referred to as Lch) and the sound content of a sound signal output from an earphone worn to the right ear (hereinafter also referred to as Rch).

DSP 120 performs processing to align the phases (in other words, match the phases, eliminate the phase difference, or make the phase difference zero) of Lch and Rch in a specific frequency band, and outputs the Lch and Rch to DAC 150. Alternatively, for example, DSP 120 performs processing to reduce the phase difference between Lch and Rch in a specific frequency band, and outputs Lch and Rch to DAC 150. For example, DSP 120 aligns the phases of Lch and Rch by performing short-time Fourier transform (more specifically, short-time fast Fourier transform (FFT)) on each of Lch and Rch, and aligns the phases of Lch and Rch in a specific frequency band by further performing inverse Fourier transform (more specifically, inverse short-time FFT).

It should be noted that in the process of aligning (matching) the phases of Lch and Rch, DSP 120 may change the phase of only Lch, may change the phase of only Rch, or may change the phases of both Lch and Rch.

In addition, making a phase difference zero is defined as substantially zero, and may have a slight phase shift rather than being completely zero.

In this way, when the first content includes a plurality of sound contents that differ from each other, DSP 120 corrects the plurality of first sound signals corresponding to the plurality of sound contents, for example, to reduce the phase difference in a specific frequency band among the plurality of first sound signals.

In addition, speakers 171 and 172 may be speakers that emit sound waves toward the eardrum, or may be bone conduction speakers.

Microphone 330 is a microphone that collects the environmental sound of the environment where the sound is output by speakers 171 and 172, and outputs a sound signal based on the collected environmental sound (hereinafter also referred to as an environmental sound signal) to CPU 130 via communication IF 180.

For example, CPU 130 obtains an environmental sound signal based on the environmental sound from microphone 330 via communication IF 180, generates additional information (second content) so as to cause a component of a specific frequency band in the environmental sound signal to be a second sound signal, and stores it in memory 143.

For example, DSP 120 enhances the signal level of a specific frequency band in the environmental sound signal obtained from microphone 330 via communication IF 180 based on highlighted information 203.

In addition, DSP 120, for example, superimposes the enhanced environmental sound signal and the sound source signal obtained from storage device 320, and outputs the superimposed signal to DAC 150. That is, the environmental sound signal is an example of the second sound signal, and is used so as to be superimposed on the sound source signal in the same way as the additional signal described above.

For example, when sound signal processing device 104 is an earphone, microphone 330 is housed in the housing of the earphone.

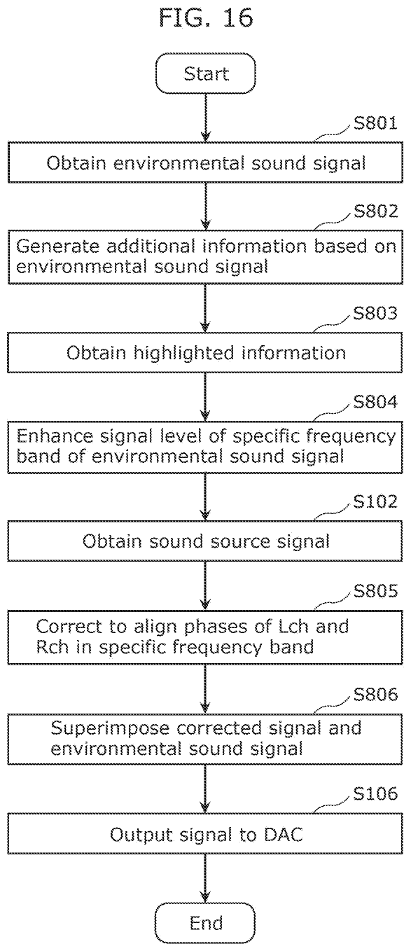

FIG. 16 is a flowchart showing the processing procedure of sound signal processing device 104 according to Variation 7 of Embodiment 1. Specifically, FIG. 16 is a flowchart showing the processing procedure of DSP 120 included in sound signal processing device 104 according to Variation 7.

First, CPU 130 obtains an environmental sound signal from microphone 330 (S801).

Next, CPU 130 generates additional information based on the environmental sound signal (S802). The additional information may be, for example, information indicating an environmental sound signal, or information indicating a signal that includes only a specific frequency band of the environmental sound signal by applying a narrow band filter to the environmental sound signal. Here, a description will be given assuming that CPU 130 generates additional information indicating an environmental sound signal and stores it in memory 143.

It should be noted that sound signal processing device 104 may include a filter circuit that functions as a narrowband filter.

Next, DSP 120 obtains, from CPU 130, highlighted information 203 that CPU 130 obtained from memory 143 (S803).

Next, DSP 120 applies a narrowband filter to the environmental sound signal and enhances the signal level of the specific frequency band based on highlighted information 203 (S804).

It should be noted that sound signal processing device 104 may include a filter circuit that functions as a narrowband filter.

Next, DSP 120 reads out the sound content from storage device 320 to obtain a sound source signal that is a sound signal based on the sound content (S102). For example, DSP 120 obtains Lch and Rch as sound source signals.

Next, DSP 120 generates a signal (correction signal) by correcting at least one of Lch or Rch so that the phases of Lch and Rch in the specific frequency band are aligned (S805). It should be noted that DSP 120 may correct at least one of Lch or Rch so as to reduce the phase difference between Lch and Rch in a specific frequency band.

Next, DSP 120 generates a signal (superimposed signal) by superimposing the corrected signal (correction signal) and the environmental sound signal (S806).

Next, DSP 120 outputs the generated signal (superimposed signal) to DAC 150 (S106).

The superimposed signals corresponding to Lch and Rch are transmitted from DAC 150 to speakers 171 and 172 via amplifier 160. Speakers 171 and 172 output sound based on the signal.

### [Effects, Etc.]

As described above, the sound signal processing method according to one aspect of the present disclosure includes: adjusting (for example, step S103 to step S104) a signal level of a second sound signal (for example, an additional signal) corresponding to a second content according to a signal level of a specific frequency band in a first sound signal (for example, a sound source signal) corresponding to a first content, the second sound signal including a component of the specific frequency band; and superimposing (for example, step S105) and outputting (for example, step S106) the first sound signal and the second sound signal adjusted.

According to this, it is possible to output sound in a specific frequency band that is effective for improving dementia and the like, along with the sound based on the first sound signal corresponding to the first content such as music. For that reason, since the user can hear the sound in the specific frequency band along with the music, it is possible to prevent the user from feeling uncomfortable due to the sound in the specific frequency band. That is, according to the sound signal processing method according to one aspect of the present disclosure, sound in a specific frequency band can be suitably output to a target (for example, a user who listens to the sound output by the sound signal processing method).

For example, in the adjustment described above, when the signal level of the specific frequency band in the first sound signal is higher than a threshold (for example, Yes in step S201), the signal level of the second sound signal is increased by a predetermined ratio to the signal level of the specific frequency band in the first sound signal (for example, step S104), and when the signal level of the first sound signal is lower than or equal to the threshold (for example, No in step S201), the signal level of the second sound signal is set to a predetermined signal level (for example, step S202 and step S104). In Variation 1 described above, DSP 120 calculates the envelope of the sound source signal (first sound signal), and if the calculated envelope is larger than the threshold, DSP 120 directly multiplies the value of the envelope by the second sound signal to increase the signal level of the second sound signal at a predetermined ratio. On the other hand, if the calculated envelope is less than or equal to the threshold, DSP 120 multiplies the value of the envelope by the second sound signal as a value corresponding to a predetermined signal level to set the signal level of the second sound signal to a predetermined signal level.

According to this, if the signal level of a specific frequency band in the first sound signal is high to a certain extent, the signal level of the second sound signal will also increase accordingly, so even if the signal level of the second sound signal is increased, it is possible to prevent the user from feeling uncomfortable due to sounds in the frequency band. In addition, if the signal level of the specific frequency band in the first sound signal is low to a certain extent, the signal level of the second sound signal can be set to a predetermined level to prevent the signal level of the second sound signal from becoming too low and becoming ineffective against dementia and the like.

In addition, for example, the first content includes a plurality of sound contents that are different from each other, and the sound signal processing method further includes correcting a plurality of first sound signals corresponding to the plurality of sound contents to reduce a phase difference in the specific frequency band among the plurality of first sound signals (for example, step S805).

According to this, by correcting to reduce the phase difference of a specific frequency band in each of a plurality of first sound signals (for example, Rch and Lch described above), when a second sound signal is superimposed on each of them, it is possible to bring the signal level of a specific frequency band closer to each other. Here, for example, there is a problem that when a user is made to listen to a sound in a specific frequency band from both ears using earphones or the like, if there is a phase difference between the sound emitted from the speaker worn to the right ear and the sound emitted from the speaker worn to the left ear, it is difficult to obtain the effect of improving dementia and the like. As such, for example, when the speakers included in the sound signal processing device are realized by earphones or the like such as speakers 171 and 172, by reducing the phase difference between the sound emitted from speaker 171 worn to the left ear and the sound emitted from speaker 172 worn to the right ear, a sound that is effective in improving dementia and the like is output.

In addition, for example, the sound signal processing method according to one aspect of the present disclosure further includes: obtaining an environmental sound signal based on an environmental sound (for example, step S801), and generating the second content to cause a component of the specific frequency band in the environmental sound signal to be the second sound signal (for example, step S802).

According to this, even if the first sound signal does not include a signal in a specific frequency band, additional information 200 can be generated using the environmental sound, and the second sound signal can be superimposed on the first sound signal and it can be output. In addition, for example, since the second sound signal is generated based on the environmental sound, even if the sound in a specific frequency band becomes louder, the sound is close to the environmental sound, so it is possible to suppress the discomfort caused by being different from the environmental sound, and it is possible to prevent the user from feeling uncomfortable due to the sound in the specific frequency band.

In addition, for example, in the adjustment described above, processing is further performed to increase the signal level of the frequency band of the overtone of the specific frequency band (for example, step S401).

It is known that overtones in specific frequency bands are also effective in improving dementia and the like, just like a sound in a specific frequency band. For that reason, according to this, a sound that is more effective in improving dementia and the like is output. In addition, for example, a small speaker or the like may not be able to output sound in a specific frequency band, which is a low frequency band of about 40 Hz, for example. Even in such a case, there is a high possibility that overtones will be output, so the effect of improving dementia and the like can be expected.

It should be noted that the adjustment may be made to increase the signal level of the frequency band of the overtone of the specific frequency band in the first sound signal, or the adjustment may be made to increase the signal level of the frequency band of the overtone of the specific frequency band in the second sound signal.

In addition, for example, the sound signal processing method according to one aspect of the present disclosure further includes obtaining control information (for example, amplitude value information 201) indicating the signal level of the specific frequency band, wherein in the adjustment described above, the signal level of the specific frequency band is controlled based on the control information (for example, step S501).

According to this, the user can listen to the sound in the specific frequency band at the user's desired sound volume.

In addition, for example, the sound signal processing method according to one aspect of the present disclosure further includes obtaining biological information (for example, pNN information 202) of a user, wherein in the adjustment described above, the signal level of the specific frequency band is controlled based on the biological information (for example, step S602).

According to this, for example, by determining whether the user is comfortable based on biological information and adjusting the signal level of the second sound signal based on the determination result, the user can easily listen to comfortable sounds without having to make adjustments himself/herself.

In addition, for example, the sound signal processing method according to one aspect of the present disclosure further includes: obtaining (for example, step S701) an output sound signal based on an output sound output (more specifically, the sound output from speakers 170, 171, and 172) in the outputting; and controlling (for example, step S702 to step S704) the signal level of the specific frequency band based on the output sound signal.

Depending on the installation environment of speaker 170 that outputs the sound output by the sound signal processing method, for example, in the case of earphones, according to how the earphones are worn by the user, speaker 170 may not output sound in a specific frequency band at an expected sound volume. As such, as described above, for example, the sound actually output from speaker 170 is collected by microphone 190, and the signal level of the specific frequency band is adjusted using a microphone signal based on the collected sound. According to this, suitable sound is output from speaker 170.

In addition, the program according to one aspect of the present disclosure is, for example, a program that causes a computer to execute the sound signal processing method according to one aspect of the present disclosure.

In addition, a sound signal processing device according to an aspect of the present disclosure includes a processor and memory, wherein using the memory, the processor: adjusts a signal level of a second sound signal corresponding to a second content according to a signal level of a specific frequency band in a first sound signal corresponding to a first content, the second sound signal including a component of the specific frequency band; and superimposes and outputs the first sound signal and the second sound signal adjusted.

According to this, the same effects as the sound signal processing method according to one aspect of the present disclosure are achieved.

It should be noted that the processor here is, for example, at least one of DSP 120 or CPU 130, and may be achieved only with DSP 120, only CPU 130, or DSP 120 and CPU 130. In addition, the memory here includes, for example, memories 140, 141, 142, and 143, but it may be realized by storage device 110, or may be realized by memories 140, 141, 142, and 143, and storage device 110. In addition, using memory means, for example, that a processor performs various processes using programs and information stored in the memory.

### Embodiment 2

Subsequently, a sound signal processing device according to Embodiment 2 will be described. It should be noted that in the following, differences from the above-described first embodiment and each variation will be mainly explained, and substantially the same configurations will be given the same reference numerals, and the explanation may be partially simplified or omitted.

The sound signal processing device according to Embodiment 2 adjusts the signal level of the first sound signal in a specific frequency band instead of superimposing the second sound signal on the first sound signal. In addition, correction is performed to reduce the phase difference between the plurality of first sound signals, each of which has its signal level adjusted in a specific frequency band. According to this, since the phase difference between the plurality of sound signals is reduced, it is possible to easily obtain the effect of improving dementia and the like.

### [Configuration]

First, the configuration of the sound signal processing device according to Embodiment 2 will be described.

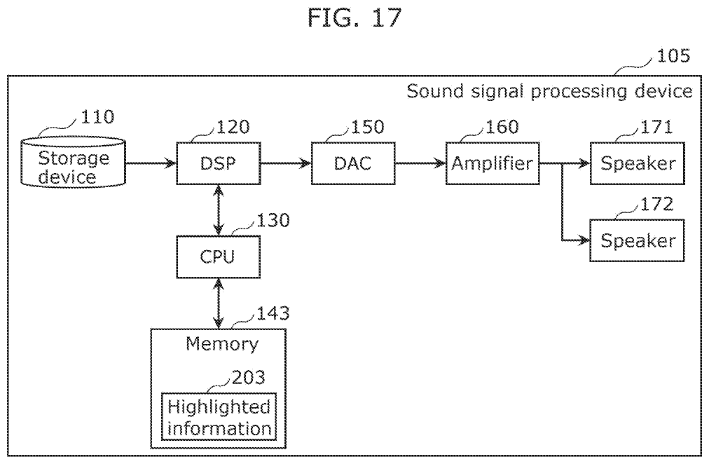

FIG. 17 is a block diagram showing the configuration of sound signal processing device 105 according to Embodiment 2.

Sound signal processing device 105 includes storage device 110, DSP 120, CPU 130, memory 143, DAC 150, amplifier 160, speaker 171, and speaker 172.

For example, DSP 120 performs processing to increase the signal level of a specific frequency band of the sound signal obtained from storage device 110 to a predetermined level based on highlighted information 203.

For example, both sound signal processing device 100 and sound signal processing device 105 perform processing to adjust (more specifically, raise) the signal level of a specific frequency band with respect to the obtained sound signal (first sound signal), and output it.

Sound signal processing device 100 superimposes a signal (second sound signal) containing only a specific frequency band on the obtained sound signal (first sound signal) to adjust the signal level of the specific frequency band in the signal to be output.

On the other hand, sound signal processing device 105 adjusts the signal level of the specific frequency band in the signal to be output by enhancing the signal in the specific frequency band with respect to the obtained sound signal (first sound signal).

Specifically, DSP 120 adjusts, to increase, the signal level of a specific frequency band of a plurality of first sound signals corresponding to a plurality of sound contents in a first content including a plurality of sound contents different from each other. In addition, DSP 120 corrects a plurality of first sound signals adjusted corresponding to the plurality of sound contents so as to reduce the phase difference in a specific frequency band among the plurality of first sound signals adjusted. In addition, DSP 120 outputs a plurality of corrected first sound signals.

### <Processing Procedure>

Subsequently, the processing procedure of sound signal processing device 105 will be explained.

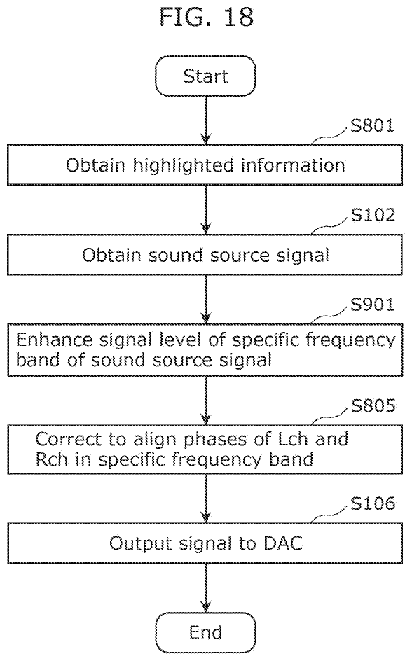

FIG. 18 is a flowchart showing the processing procedure of sound signal processing device 105 according to Embodiment 2. Specifically, FIG. 18 is a flowchart showing the processing procedure of DSP 120 included in sound signal processing device 105.

First, DSP 120 obtains, from CPU 130, highlighted information 203 that CPU 130 obtained from memory 143 (S801).

Next, DSP 120 reads out a sound content from storage device 110 to obtain a sound source signal that is a sound signal based on the sound content (S102). For example, DSP 120 obtains Lch and Rch as sound source signals.

Next, DSP 120 applies a narrowband filter to the sound source signal and enhances the signal level of a specific frequency band based on highlighted information 203 (S901). For example, DSP 120 identifies the specific frequency band for each of Lch and Rch, and increases the signal level for each of Lch and Rch according to a predetermined signal level indicated by highlighted information 203.

It should be noted that sound signal processing device 105 may include a filter circuit that functions as a narrowband filter.

Next, DSP 120 generates a signal (correction signal) by correcting at least one of Lch or Rch so that the phases of Lch and Rch in a specific frequency band are aligned (S805). It should be noted that DSP 120 may correct at least one of Lch or Rch so as to reduce the phase difference between Lch and Rch in a specific frequency band.

Next, DSP 120 outputs a signal (correction signal) in which the phase difference is corrected to zero to DAC 150 (S106).

The correction signal is transmitted from DAC 150 to speakers 171 and 172 via amplifier 160. Speakers 171 and 172 output sound based on the correction signal.

### [Effects, Etc.]

As described above, a sound signal processing method according to another aspect of the present disclosure includes: adjusting to increase a signal level of a specific frequency band of each of a plurality of first sound signals (for example, Lch and Rch) in a first content including a plurality of sound contents that are different from each other, the plurality of first sound signals corresponding to the plurality of sound contents (for example, step S901); correcting each of the plurality of first sound signals adjusted to reduce a phase difference of the specific frequency band among the plurality of first sound signals adjusted, the plurality of first sound signals adjusted corresponding to the plurality of sound contents (for example, step S804); and outputting the plurality of first sound signals corrected (for example, step S106).

In recent years, for example, earphones and the like may emit different sounds between the sound output to the right ear and the sound output to the left ear for reasons such as enhancing the realism of the sound and the like. However, it is known that, for example, if the sound in a specific frequency band is different according to whether it is output to the right ear or the left ear, it is difficult to obtain an effect on dementia or the like. As such, by correcting to reduce the phase difference between specific frequency bands among the plurality of first sound signals (for example, Rch and Lch described above), it is possible to reduce the difference in the signal level between specific frequency bands. For that reason, sounds that are effective in improving dementia and the like are output. That is, according to the sound signal processing method according to another aspect of the present disclosure, sounds in a specific frequency band can be suitably output to a target.

In addition, the program according to one aspect of the present disclosure may be a program that causes a computer to execute the sound signal processing method according to another aspect of the present disclosure described above.

In addition, a sound signal processing device according to another aspect of the present disclosure includes: a processor and memory, wherein using the memory, the processor: adjusts, to increase, a signal level of a specific frequency band of each of a plurality of first sound signals in a first content including a plurality of sound contents that are different from each other, the plurality of first sound signals corresponding to the plurality of sound contents; corrects each of the plurality of first sound signals adjusted to reduce a phase difference of the specific frequency band among the plurality of first sound signals adjusted, the plurality of first sound signals adjusted corresponding to the plurality of sound contents; and outputs the plurality of first sound signals corrected.

According to this, the same effects as the sound signal processing method according to another aspect of the present disclosure are achieved.

It should be noted that the processor here is, for example, at least one of DSP 120 or CPU 130, and may be achieved with only DSP 120, only CPU 130, or DSP 120 and CPU 130. In addition, the memory here is, for example, memory 143, but it may be realized by storage device 110, or memory 143 and storage device 110.

### OTHER EMBODIMENTS

Although each of embodiments and variations has been described above, the present disclosure is not limited to each of the embodiments and variations described above.

For example, each of the embodiments and variations described above may be realized in any combination. For example, in the configuration (processing procedure) of Variation 2 described above, the sound signal processing device according to the present disclosure may change the value of the corresponding signal to the threshold if the corresponding signal is smaller than the threshold. More specifically, in the processing procedure shown in FIG. 6 for the sound signal processing device according to the present disclosure, the processing similar to step S201 and step S202 shown in FIG. 5 may be added between step S301 and step S302 shown in FIG. 6. In addition, for example, the functions of the sound signal processing device according to Variation 3 described above (for example, overtone multiplication processing) may be achieved in combination with the functions of the sound signal processing device according to Embodiment 1 (for example, envelope multiplication processing) or may be realized in combination with the functions of other sound signal processing devices. In addition, for example, the functions of the sound signal processing device according to Variation 4 described above (for example, signal level adjustment processing based on control information) may be realized in combination with the functions of other sound signal processing devices. In addition, for example, the functions of the sound signal processing device according to Variation 5 described above (for example, signal level adjustment processing based on biological information) may be realized in combination with the functions of other sound signal processing devices.

In addition, for example, based on at least one of the control information or biological information, the signal level of a specific frequency band in the first sound signal may be adjusted or the signal level of a specific frequency band in the second sound signal may be adjusted. That is, based on at least one of the control information or biological information, the relative relationship between the signal levels of the first sound signal and the second sound signal in the specific frequency band may be adjusted. Alternatively, the signal level of a specific frequency band in the superimposed signal may be adjusted.

In addition, for example, based on at least one of the control information or biological information, the signal level of a frequency band of an overtone of a specific frequency band in the first sound signal may be adjusted, and the signal level of a frequency band of an overtone of a specific frequency band in the second sound signal may be adjusted. That is, based on at least one of the control information or biological information, the relative relationship between the signal levels of the first sound signal and the second sound signal in the frequency band of the overtone of the specific frequency band may be adjusted. Alternatively, the signal level of a frequency band of an overtone of a specific frequency band in the superimposed signal may be adjusted.

In addition, for example, when the signal level of the first sound signal is zero (that is, no sound), the second sound signal may or may not be superimposed. In addition, for example, when the signal level of the first sound signal is zero, the signal level of the specific frequency band in the first sound signal may or may not be increased.

In addition, the configuration of the sound signal processing device according to each of the embodiments and variations described above is merely an example. For example, the sound signal processing device may include a component not shown, such as a D/A converter or a filter.

In addition, in each of the embodiments described above, the plurality of first sound signals corresponding to the plurality of sound contents have been described by exemplifying two first sound signals as Lch and Rch, for example. However, the plurality of first sound signals may be three or more.

In addition, in the embodiments described above, the sound signal processing device may be realized as a plurality of devices (that is, a system), or may be realized as a single device. When the sound signal processing device is realized by a plurality of devices, the functional components included in the sound signal processing device may be distributed to the plurality of devices in any manner. For example, a mobile terminal may include some or all of the functional components included in the sound signal processing device.

In addition, the communication method between devices in each of the embodiments and variations described above is not particularly limited. When two devices communicate in each of the embodiments and variations described above, a relay device (not shown) may be interposed between the two devices.

In addition, the order of processes described in each of the embodiments and variations described above is an example. The order of a plurality of processes may be changed, and a plurality of processes may be executed in parallel. In addition, a process performed by a particular processing unit may be performed by another processing unit. In addition, some of the digital signal processing described in each of the embodiments and variations described above may be realized by analog signal processing.

In each of the embodiments and variations described above, each component may be realized by executing a software program suitable for each component. Each component may be realized by a program execution unit such as a CPU or processor reading and executing a software program recorded on a recording medium such as a hard disk or semiconductor memory.

In addition, each component may be realized by hardware. For example, each component may be a circuit (or integrated circuit). These circuits may constitute a single circuit as a whole, or they may be separate circuits. In addition, each of these circuits may be a general-purpose circuit or a dedicated circuit.

The general or specific aspects of the present disclosure may be realized using a system, a method, an integrated circuit, a computer program, or a computer-readable recording medium such as a CD-ROM. In addition, they may be realized using any combination of systems, methods, integrated circuits, computer programs, and recording media. For example, the present disclosure may be implemented as a method to be executed by a computer such as a sound signal processing device or a portable terminal, or as a program for causing a computer to execute such a method. In addition, the present disclosure may also be realized as a computer-readable non-transitory recording medium having such a program recorded thereon. It should be noted that the program herein includes an application program for making a general-purpose portable terminal function as the portable terminal of each of the embodiments and variations described above.

In addition, forms obtained by applying various modifications to each of the embodiments and variations conceived by a person skilled in the art or forms realized by arbitrarily combining the components and functions in each of the embodiments and variations without departing from the spirit of the present disclosure are also included in this disclosure.

### INDUSTRIAL APPLICABILITY

The sound signal processing device of the present disclosure can be applied to a device that outputs sounds that can improve dementia and the like.

1. A sound signal processing method comprising:
adjusting a signal level of a second sound signal corresponding to a second content according to a signal level of a specific frequency band in a first sound signal corresponding to a first content, the second sound signal including a component of the specific frequency band; and
superimposing and outputting the first sound signal and the second sound signal adjusted.

2. The sound signal processing method according to claim 1,
wherein in the adjusting,
when the signal level of the specific frequency band in the first sound signal is higher than a threshold, the signal level of the second sound signal is increased by a predetermined ratio to the signal level of the specific frequency band in the first sound signal, and
when the signal level of the first sound signal is lower than or equal to the threshold, the signal level of the second sound signal is set to a predetermined signal level.

3. The sound signal processing method according to claim 1,
wherein the first content includes a plurality of sound contents that are different from each other, and
the sound signal processing method further comprises correcting a plurality of first sound signals corresponding to the plurality of sound contents to reduce a phase difference in the specific frequency band among the plurality of first sound signals, each of the plurality of first sound signals being the first sound signal.

4. The sound signal processing method according to claim 1, further comprising:
obtaining an environmental sound signal based on an environmental sound; and
generating the second content to cause a component of the specific frequency band in the environmental sound signal to be the second sound signal.

5. A sound signal processing method comprising:
adjusting, to increase, a signal level of a specific frequency band of each of a plurality of first sound signals in a first content including a plurality of sound contents that are different from each other, the plurality of first sound signals corresponding to the plurality of sound contents;
correcting each of the plurality of first sound signals adjusted to reduce a phase difference in the specific frequency band among the plurality of first sound signals adjusted, the plurality of first sound signals adjusted corresponding to the plurality of sound contents; and
outputting the plurality of first sound signals corrected.

6. The sound signal processing method according to claim 1,
wherein in the adjusting, a signal level of a frequency band of an overtone of the specific frequency band is further controlled to increase.

7. The sound signal processing method according to claim 1, further comprising:
obtaining control information indicating the signal level of the specific frequency band,
wherein in the adjusting, the signal level of the specific frequency band is controlled based on the control information.

8. The sound signal processing method according to claim 1, further comprising:
obtaining biological information of a user,
wherein in the adjusting, the signal level of the specific frequency band is controlled based on the biological information.

9. The sound signal processing method according to claim 1, further comprising:
obtaining an output sound signal based on an output sound output in the outputting; and
controlling the signal level of the specific frequency band based on the output sound signal.

10. A non-transitory computer-readable recording medium having recorded thereon a program for causing a computer to execute the sound signal processing method according to claim 1.

11. A sound signal processing device comprising:
a processor; and
memory,
wherein using the memory, the processor:
adjusts a signal level of a second sound signal corresponding to a second content according to a signal level of a specific frequency band in a first sound signal corresponding to a first content, the second sound signal including a component of the specific frequency band; and
superimposes and outputs the first sound signal and the second sound signal adjusted.

12. A sound signal processing device comprising:
a processor; and
memory,
wherein using the memory, the processor:
adjusts, to increase, a signal level of a specific frequency band of each of a plurality of first sound signals in a first content including a plurality of sound contents that are different from each other, the plurality of first sound signals corresponding to the plurality of sound contents;
corrects each of the plurality of first sound signals adjusted to reduce a phase difference of the specific frequency band among the plurality of first sound signals adjusted, the plurality of first sound signals adjusted corresponding to the plurality of sound contents; and
outputs the plurality of first sound signals corrected.
